(19) United States
(12) Patent Application Publication
(10) Pub. No.: US 2007/0179483 A1
(43) Pub. Date: Aug. 2, 2007

(54) Method and device for producing a closed curved cut

(75) Inventors: Dirk Muhlhoff, Kunitz (DE); Mario Gerlach, Altenberga (DE); Markus Sticker, Jena (DE); Carsten Lang, Bad Kostritz (DE); Mark Bischoff, Elleben OT Riechheim (DE); Michael Bergt, Jena (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: CARL ZEISS MEDITEC AG, Jena (DE)

(21) Appl. No.: 10/565,723

(22) PCT Filed: Jul. 21, 2004
(86) PCT No.: PCT/EP04/08161

§ 371 Date: Aug. 11, 2006

(30) Foreign Application Priority Data
Jul. 25, 2003 (DE) 103 34 108.0

(51) Int. Cl. A61B 18/18

(52) U.S. Cl. 606/10; 606/4

(57) ABSTRACT

A method of forming a cut which encloses a partial volume within a transparent material, by generating optical breakthroughs in the material by means of laser radiation focused into the material along an optical axis in which the focal point is three-dimensionally adjusted so as to form the cut by serial arrangement of the optical breakthroughs, wherein the focal point is adjusted along a spatial spiral, which is located in the cut and extends along a main axis that is at substantially right angles to the optical axis.

The invention relates to a device for producing a cut enclosing a partial volume within a transparent material, said device comprising a source of laser radiation, which focuses laser radiation into the material to cause optical breakthroughs therein, wherein a scanning unit, which three-dimensionally adjusts the focal point, and a control unit, which controls the scanning unit, are provided so as to produce the cut by serially arranging the optical breakthroughs. The invention further relates to a method of producing a cut enclosing a partial volume within a transparent material by generating optical breakthroughs in the material by means of laser radiation, which is focused into the material along an optical axis, wherein the focal point is three-dimensionally adjusted, so as to produce the cut by serially arranging the optical breakthroughs.

Curved cuts within a transparent material are generated particularly in laser-surgical methods, especially in eye surgery. This involves focusing treatment laser radiation within the tissue, i.e. beneath the tissue surface, so as to form optical breakthroughs in the tissue.

In the tissue, several processes initiated by interaction with the laser radiation occur in a time sequence. If the power density of the radiation exceeds a threshold value, an optical breakthrough will result, generating a plasma bubble in the material. After the optical breakthrough has formed, said plasma bubble grows due to expanding gases. Subsequently, the gas generated in the plasma bubble is absorbed by the surrounding material, and the bubble disappears again. However, this process takes very much longer than the forming of the bubble itself. If a plasma is generated at a material boundary, which may quite well be located within a material structure as well, material will be removed from said boundary. This is then referred to as photo ablation. In connection with a plasma bubble which separates material layers that were previously connected, one usually speaks of photo disruption. For the sake of simplicity, all such processes are summarized herein by the term optical breakthrough, i.e. said term includes not only the actual optical breakthrough, but also the effects resulting therefrom in the material.

For high accuracy of a laser surgery method, it is indispensable to guarantee high localization of the effect of the laser beams and to avoid collateral damage to adjacent tissue as far as possible. It is, therefore, common in the prior art to apply the laser radiation in pulsed form, so that the threshold value for the power density of the laser radiation required to cause an optical breakthrough is exceeded only during the individual pulses. In this regard, U.S. Pat. No. 5,984,916 clearly shows that the spatial extent of the optical breakthrough (in this case, of the generated interaction) strongly depends on the pulse duration. Therefore, high focusing of the laser beam in combination with very short pulses allows to place the optical breakthrough in a material with great point accuracy.

The use of pulsed laser radiation has recently become established practice in ophthalmology, particularly for laser-surgical correction of visual defects. Visual defects of the eye often result from the fact that the refractive properties of the cornea and of the lens do not cause optimal focusing on the retina.

U.S. Pat. No. 5,984,916 mentioned above, as well as U.S. Pat. No. 6,110,166, describe methods of the above-mentioned type for producing cuts by means of suitable generation of optical breakthroughs, so that, ultimately, the refractive properties of the cornea are selectively influenced. A multitude of optical breakthroughs are joined such that a lens-shaped partial volume is isolated within the cornea of the eye. The lens-shaped partial volume which is separated from the remaining corneal tissue is then removed from the cornea through a laterally opening cut. The shape of the partial volume is selected such that, after removal, the shape and, thus, the refractive properties of the cornea are changed so as to have the desired correction of the visual defect. The cuts required here are curved, which makes a three-dimensional adjustment of the focus necessary. Therefore, a two-dimensional deflection of the laser radiation is combined with simultaneous adjustment of the focus in a third spatial direction.

The two-dimensional deflection of the laser radiation and the focus adjustment are both equally decisive for the accuracy with which the cut can be produced. At the same time, the speed of adjustment, which is achievable thereby, has an effect on the speed at which the required cut can be produced. Generating the cuts quickly is desirable not only for convenience or in order to save time; bearing in mind that movements of the eye inevitably occur during ophthalmological operations, quick generation of cuts additionally contributes to the optical quality of the result thus achieved and reduces the demands made on possible tracking of eye movements.

Therefore, it is an object of the invention to improve a method and an apparatus of the above-mentioned type such that the time required to generate a cut is as short as possible.

According to the invention, this object is achieved by a device of the above-mentioned type, whose control unit adjusts the focal point along a space spiral, which is located in the cut and extends along a main axis which is at substantially right angles to the optical axis. The object is further achieved by a device of the above-mentioned type, wherein the control unit adjusts the focal point along elevation lines, which are located in planes that are substantially parallel to the optical axis.

The object is further achieved by a method of the above-mentioned type, wherein the focal point is adjusted along a space spiral, which is located in the cut and extends along a main axis which is at substantially right angles to the optical axis. Thus, the main axis is the screw axis along which the spiral extends.

Finally, the invention is further solved by a method of the above-mentioned type, wherein the focal point is adjusted along elevation lines of the cut, which are located in planes that are substantially parallel to the optical axis.

Thus, the invention departs from the conventional scanning of a curved cut and effects a simultaneous cut advancement at parts of the cut which are located in different places along the optical axis. In contrast thereto, it was always known in the prior art to first cut those surface parts of a cut which are more distant on the optical axis. Analogous to ophthalmological nomenclature, this more distant surface is referred to hereinafter as the posterior surface. In the prior art, it was only after complete scanning of the posterior side of the cut that the nearer surface on the optical axis of the treatment device was cut, which surface is referred to hereinafter as the anterior surface.

According to the invention, a cut advancement is now effected alternately at the posterior and the anterior surface. This concept allows to avoid high adjustment speeds of the focal point along the optical axis, in spite of a constant cutting speed. Since such adjustment is conveniently effected by an adjustable telescope, the mechanical demands made on the optical system by the control unit according to the invention or by the method according to the invention are thus strongly reduced. Since the focal point is adjusted along a spiral or along elevation lines, the reversal points required in the prior art, which necessitated a high adjustment speed at the transition from the posterior to the anterior partial surface of the cut, are no longer present. Instead, an almost mono-frequent or very narrow-band adjustment can be worked with in the direction of the optical axis.

When generating the cut by serial arrangement of optical breakthroughs, it should be borne in mind that, in some cases, the generation of a breakthrough behind an already generated breakthrough is possible only in very poor quality and sometimes not at all, because a cut generated anteriorly on the optical axis may result in scattering effects which affect the beam quality of the laser beam, as it passes through, such that no desired breakthrough is possible posteriorly any more. Therefore, care should be taken to avoid a situation in which an anterior cut covers a posterior site at which an optical breakthrough is to be generated. This can be achieved by beginning to generate optical breakthroughs on each elevation line or on the posterior part on the spiral. In addition, it may be ensured that the main axis to which the spiral is related is not, relative to the optical axis, which also applies to the parallelism of the planes of the elevation lines and the optical axis. A deviation just great enough to cause an anterior focus trace to be located just next to the immediately adjacent posterior extension, is sufficient. Thus, the angular deviation may be very small; therefore, such deviation shall be covered by the terms “at substantially right angles” or “substantially parallel”. Thus, the main axis or the planes coincide(s) with an axis that is perpendicular to the optical axis or enclose(s) an acute angle therewith.

The scanning unit which adjusts the focal point conveniently comprises adjustable optics for adjustment along the optical axis and a deflecting unit for two-dimensional adjustment of the focal point perpendicular to the optical axis. The deflecting unit may be provided by tilting or swivelling mirrors having axes of rotation that cross each other. The axes of rotation will conveniently be selected so as to be respectively at right angles to the optical axis.

The control unit ensures suitable operation of the deflecting unit. For this purpose, for example, it may control the scanning unit with a triangle function in one direction, and with a linear function having an oscillation or a step function with a small amplitude superimposed thereon, in the other direction. Adjustment of the focal point along the optical axis may then be effected by a sinusoidal function, so that the control unit causes a resulting three-dimensional shape of the curve of the focal point in the form of an ellipse located obliquely in space or of an ellipsoid structure, the control unit ensuring that the trace of the ellipse to be cut is not covered by an area already cut anteriorly.

The control of the adjustment along the optical axis according to a sinusoidal function shows that the frequency requirements for the adjustment unit are very minor, because a sinusoidal function may formed, for example, from small-bandwidth sinus functions in a Fourier synthesis.

The invention will be explained in more detail below, by way of example and with reference to the Figures, wherein:

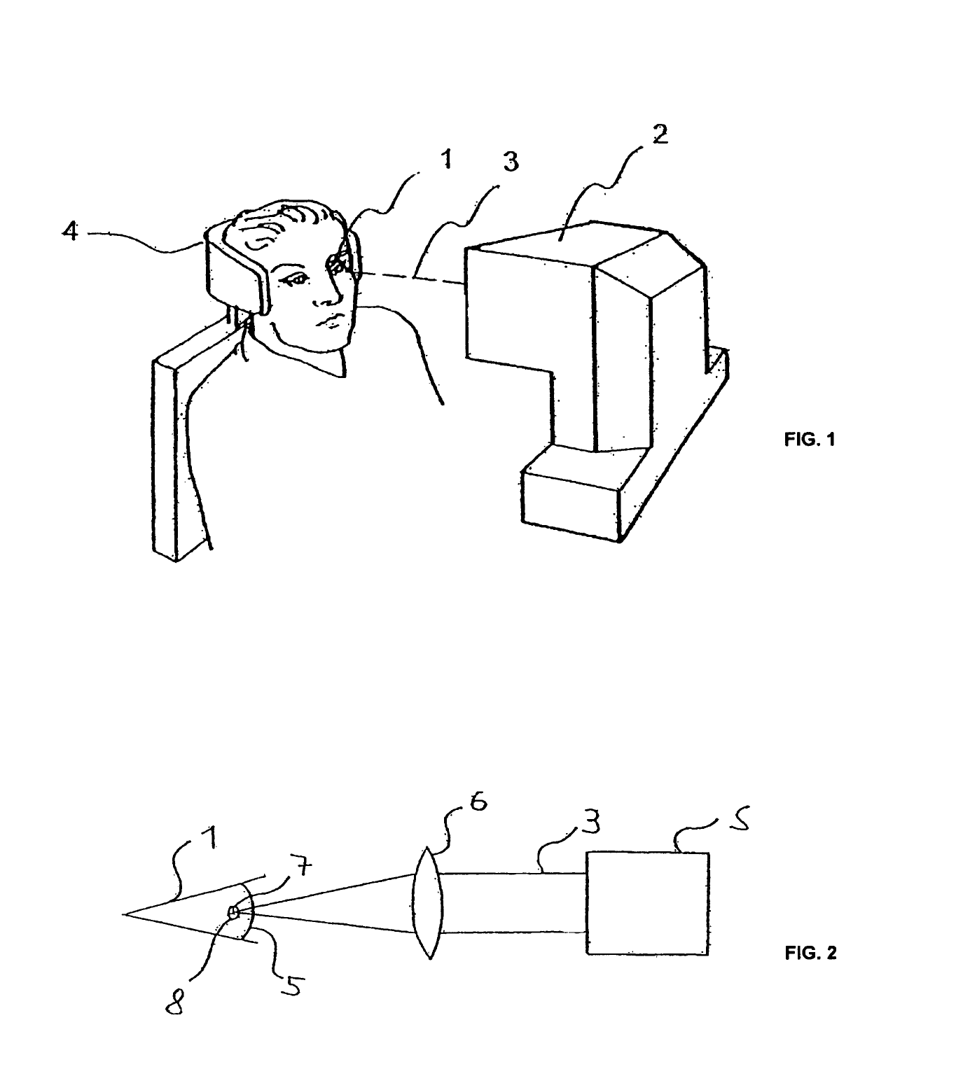

FIG. 1 shows a perspective view of a patient during a laser-surgical treatment using a laser-surgical instrument,

FIG. 2 shows the focusing of a beam onto the eye of the patient with the instrument of FIG. 1;

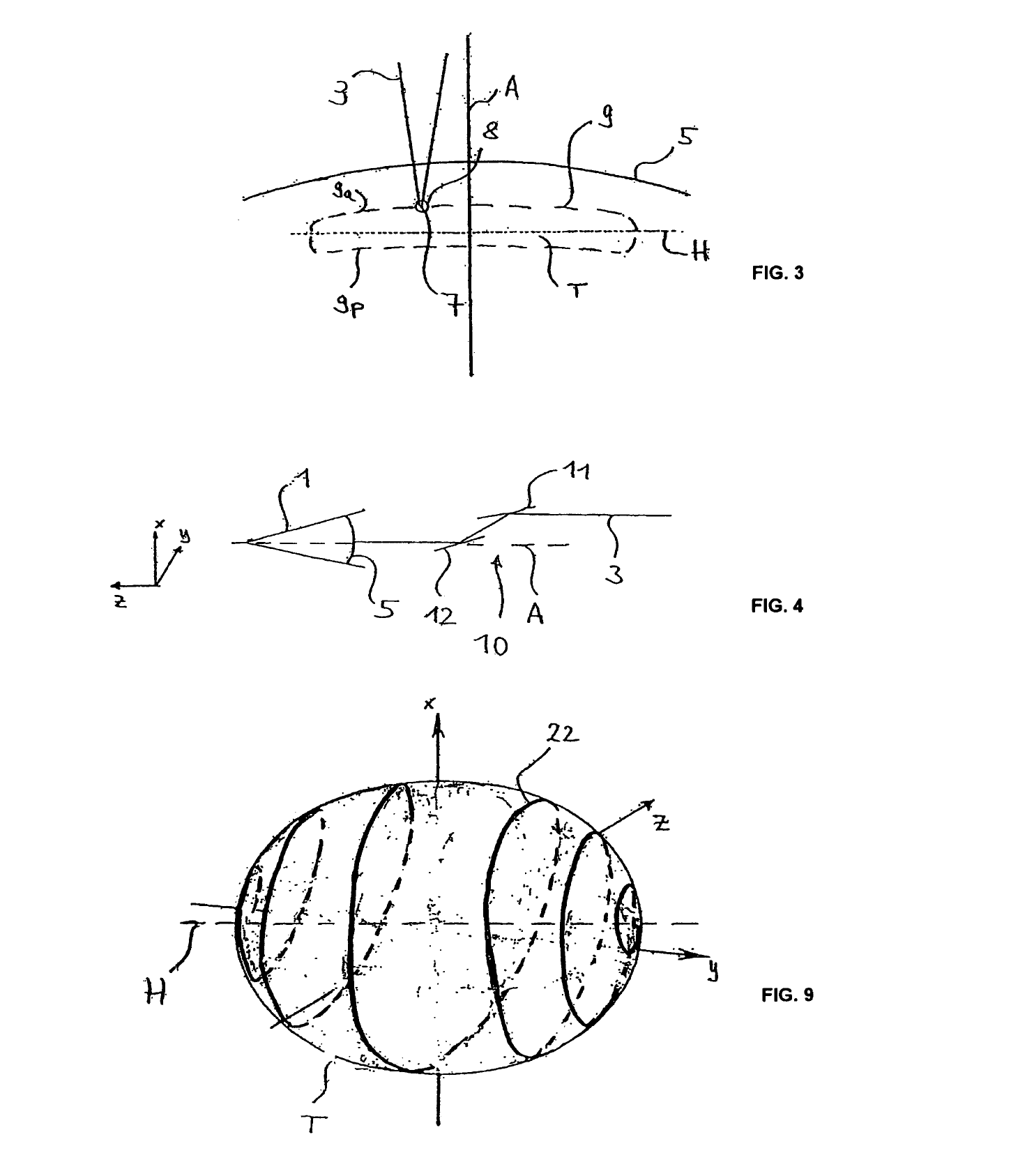

FIG. 3 shows a schematic representation illustrating a cut generated during laser-surgical treatment with the instrument of FIG. 1;

FIG. 4 shows a deflection device of the laser-surgical instrument of FIG. 1;

FIG. 5 shows an exemplary time behavior of a control function for controlling the line mirror of FIG. 4,

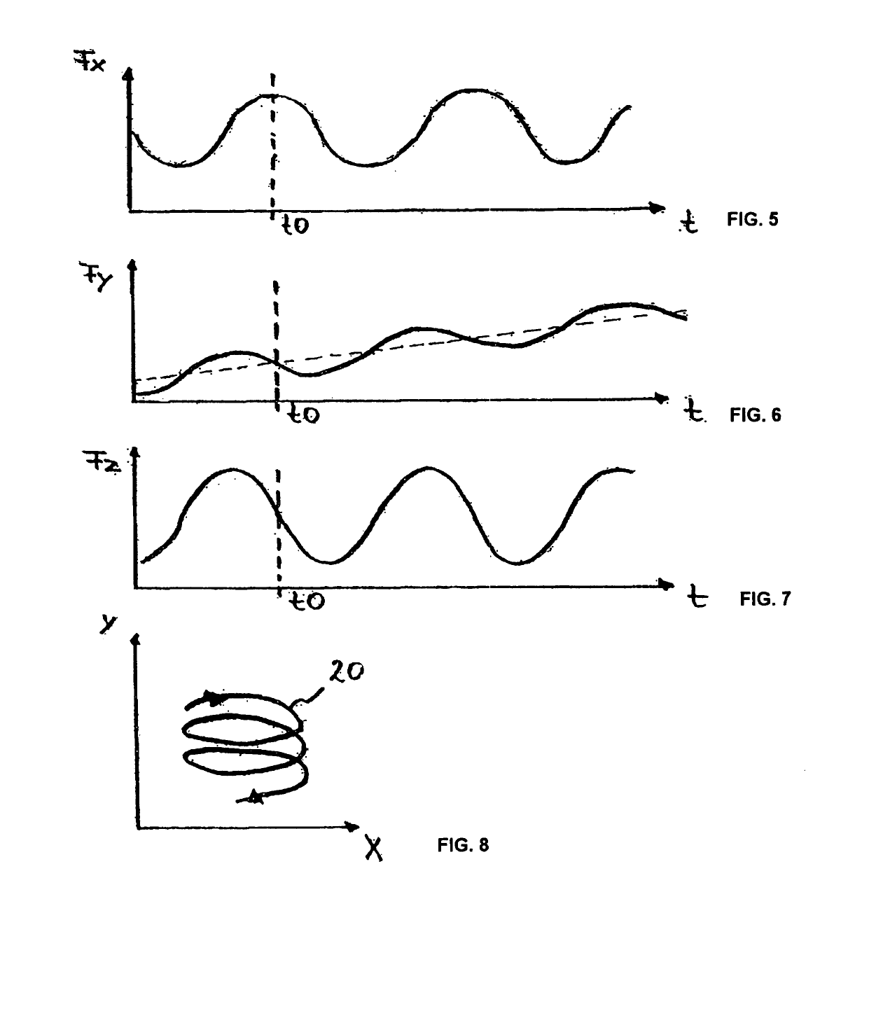

FIG. 6 shows an exemplary time sequence for the control function of the image mirror of FIG. 4,

FIG. 7 shows an exemplary time sequence for controlling the zoom optics of FIG. 2,

FIG. 8 shows views depicting how a cut is guided in the y/x- or z/y-planes of the partial volume of FIG. 3;

FIG. 9 shows a perspective view illustrating the focal point adjustment during forming of a curved, closed cut, and

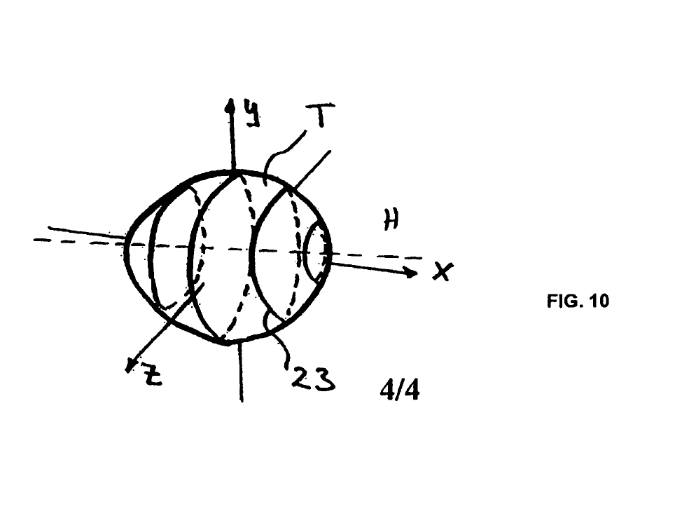

FIG. 10 shows a perspective view similar to FIG. 9.

FIG. 1 shows a laser-surgical instrument for treatment of an eye 1 of a patient, said laser-surgical instrument 2 serving to effect a refractive correction. For this purpose, the instrument 2 emits a treatment laser beam 3 onto the eye of the patient 1 whose head is immobilized in a head rest 4. The laser-surgical instrument 2 is capable of generating a pulsed laser beam 3 allowing the method described in U.S. Pat. No. 6,110,166 to be carried out.

For this purpose, as schematically shown in FIG. 2, the laser-surgical instrument 2 comprises a source of radiation S whose radiation is focused into the cornea 5 of the eye 1. A visual defect in the eye 1 of the patient is remedied using the laser-surgical instrument 2 to remove material from the cornea 5 so as to change the refractive characteristics of the cornea by a desired amount. In doing so, the material is removed from the corneal stroma, which is located beneath the epithelium and Bowman's membrane and above Decemet's membrane and the endothelium.

Material removal is effected by separating layers of tissue in the cornea, focusing the high-energy pulsed laser beam 3 by means of a telescope 6 in a focus 7 located in the cornea 5. Each pulse of the pulsed laser radiation 3 thus generates an optical breakthrough in the tissue, said breakthrough initiating a plasma bubble 8. As a result, the tissue layer separation covers a larger area than the focus 7 of the laser radiation 3. By suitable deflection of the laser beam 3, many plasma bubbles 8 are now serially arranged during treatment. The serially arranged plasma bubbles 8 then form a cut 9, which circumscribes a partial volume T of the stroma, namely the material to be removed from the cornea 5.

Due to the laser radiation 3, the laser-surgical instrument 2 operates in the manner of a surgical knife which, without injuring the upper layers of the cornea 5, directly separates material layers within the cornea 5. If the cut is guided all the way to the surface of the cornea 5 by generating further plasma bubbles 8, material of the cornea 5 isolated by the cut 9 can be pulled out laterally and thus removed.

The generation of the cut 9 by means of the laser-surgical instrument 2 is schematically shown in FIG. 3. The cut 9 is formed by serial arrangement of plasma bubbles 8 as a result of continuous displacement of the focus 7 of the pulsed focused laser beam 3.

On the one hand, lateral focus displacement according to one embodiment is effected by means of the deflecting unit 10, schematically shown in FIG. 4, which deflects the laser beam 3 about two mutually perpendicular axes, said laser beam 3 being incident on the eye 1 on an optical axis A serving as the main axis. For this purpose, the deflecting unit 10 uses a line mirror 11 as well as an image mirror 12, thus resulting in two spatial axes of deflection which are located behind each other. The point where the main beam axis and the deflection axis cross is then the respective point of deflection. On the other hand, the telescope 6 is suitably adjusted for areal focus displacement. This allows adjustment of the focus 7 along three orthogonal axes in the x/y/z coordinate system schematically shown in FIG. 4. The deflecting unit 10 adjusts the focus in the x/y plane, with the line mirror allowing adjustment of the focus in the x-direction and the image mirror allowing adjustment of the focus in the y-direction. In contrast thereto, the telescope 6 acts on the z-coordinate of the focus 7. All components of the instrument 2 are controlled by a control unit which is preferably incorporated into the instrument.

If a cut as shown in FIG. 3 is vaulted in the same direction as the corneal surface, this may be achieved with an optical system whose image field curvature is similar to the curvature of the cornea, without the guididing of the focus 7 having to reflect this.

As is evident from FIG. 3, the treatment laser beam 3 is incident on the eye 1 along or on the optical axis A. Thus, the partial volume T enclosed by the cut 9 comprises boundary surfaces which are located along the optical axis A at different distances from the instrument 2. The cut 9 can be divided into an anterior partial surface 9a as well as into a posterior partial surface 9p, which is located behind the anterior partial surface 9a on the optical axis. In order to generate the cut 9, the focus 7 is cyclically adjusted from the posterior partial surface 9p to the anterior partial surface 9a, and back. Thus, the cut 9 is generated simultaneously at the front and rear surfaces of the partial volume T.

In a first embodiment, the focus 7 is adjusted along a spatial spiral related to a main axis H. The control signals emitted by the control unit to the deflecting unit 10 as well as to the zoom optics 6 are shown in FIGS. 5, 6, and 7 by way of example. FIG. 8 shows paths of the focus 7 in two planes. FIG. 9 illustrates the spatial spiral scanned by the focus 7 in a perspective view.

As FIG. 9 shows, the cut 9 is generated for isolation of the partial volume T, by adjusting the focus 7 along a spatial spiral 22, along which the plasma bubbles 8 form the cut 9. For simplification, FIG. 9 shows the distance between individual spiral windings very much greater than required for assembling the closed cuts 9 from the plasma bubbles 8. As is evident from FIG. 9, the main axis H, along which the spatial spiral 22 extends, is at an acute angle to an axis located at right angles to the optical axis A, which optical axis A coincides with the coordinate axis z as shown in FIG. 9. Thus, the curve of the focus 7 alternately scans a line (shown in broken lines in FIG. 9) which is located in the posterior partial surface 9p and then a line (shown in solid lines in FIG. 9) which is part of the anterior partial surface 9a.

In order to adjust the focus 7 along the spatial spiral 22, the control unit of the instrument 2 applies the sinus function Fx shown in FIG. 5 to the line mirror 11. Thus, the line mirror effects a reciprocating tilting oscillation.

In addition to the control function Fx, the image mirror 12, which causes deflection in the y-direction, is controlled by a control function Fy (cf. FIG. 6), which corresponds to a slow linear increase, onto which an oscillation having a small amplitude is superimposed. At the time t0, at which the control function Fx has a maximum, the control function Fy exhibits a value which corresponds exactly to the linear increase (shown in broken lines in FIG. 6). If Fx is located on a mean value, Fy exhibits the maximum distance from the linear increase. The frequencies which occur in the control function Fy and which the image mirror 12 has to satisfy are about 1/1000 of those occurring in the function Fx.

Now, in order to keep already cut tissue of anterior layers from disturbing any posterior generation of a plasma bubble 8, i. e. to keep the laser beam 3 from contacting already cut areas, the oscillation superimposed on the linear increase is provided in the control function Fy. This results in the focus 7 being located in a y-coordinate during the return movement of the line mirror, which y-coordinate is part of an area in which the associated anterior partial surface 9a has not yet been cut.

At the same time, adjustment along the optical axis A, i. e. in the z-direction in FIG. 9, is effected according to a sinusoidal movement, which has a mean value at the time t0 and at further times, at which Fx has a maximum value and Fy has a value corresponding to the linear increase. The control function Fz for the zoom optics 6 is thus in phase with the oscillation of the control function Fy for the image mirror 12.

The sinusoidal movement of the zoom optics 6 results in the three-dimensional shape of the path shown in FIG. 9 in the form of an ellipsoid structure arranged obliquely in space, wherein it is ensured that anterior path curves do not cover the posterior parts of the path curve which are to be cut next.

Depending on the cut 9 to be formed, different control functions Fx, Fy, Fz are provided. However, what they all have in common is that the cut 9 is formed simultaneously at the front and rear surfaces and that this requires a slow adjustment speed in the z-direction.

FIG. 8 schematically shows a detail of a path curve 20 of the focus 7 in a projection in the y/x plane. In a second embodiment, the focus 9 is adjusted, as shown in FIG. 10, along elevation lines 23, which are oriented relative to a main axis H that is perpendicular to the optical axis A, i.e. which are located in one plane relative to said main axis H. In said embodiment, the main axis H is perpendicular to the optical axis A which is identical with the z-axis in FIG. 10, so that the elevation lines 23 define planes which are parallel to the optical axis A, which is to be considered the main axis of incidence.

In a first variant, the focus 7 is adjusted by the deflecting unit 10 and the telescope 6 under the control of the control unit so as to scan each elevation line such that first the posterior part, i.e. the portion located in the posterior partial surface 9p, and then the anterior part, i. e. the portion located in the anterior partial surface 9a, of the elevation line is scanned. This ensures that no anterior plasma bubble 8 shades any location located on the posterior partial surface 9p, on which a plasma bubble 8 is to be generated. Alternatively or additionally, according to a second variant, the plane of each elevation line 23 may be slightly tilted relative to the optical axis A. According to one embodiment, said tilting is selected such that, in projection along the optical axis A, plasma bubbles 8 located on the posterior portion of an elevation line are arranged adjacent to the plasma bubbles 8 being generated on the anterior part of the elevation line. According to a variant, there may even be a certain spacing between said plasma bubbles.

1-11. (canceled)

12. A method of forming a cut which encloses a partial volume within a transparent material, by generating optical breakthroughs in the material by application of laser radiation focused into the material at a focal point substantially along an optical axis, comprising the steps of:
adjusting the focal point three-dimensionally to form the cut by sequential arrangement of the optical breakthroughs; and
adjusting the focal point along a spatial spiral, which is located in a desired location of the cut and extends along a main axis that is at substantially right angles to the optical axis.

13. The method as claimed in claim 12, wherein the spiral is begun in a part of the transparent material which is posteriorly located substantially along the optical axis.

14. The method as claimed in claim 13, wherein the main axis is located such that posterior parts of the transparent material are not obscured by previously generated optical breakthroughs located in anterior parts of the transparent material.

15. A method of forming a cut which encloses a partial volume within a transparent material, by generating optical breakthroughs in the material by application of laser radiation focused into the material at a focal point along an optical axis, comprising the steps of:
adjusting the focal point three-dimensionally to form the cut by sequential arrangement of the optical breakthroughs; and
adjusting the focal point along elevation lines in the desired location of the cut, which are located in planes that are substantially parallel to the optical axis.

16. The method as claimed in claim 15, wherein each elevation line is begun on a part which is posterior to the optical axis.

17. The method as claimed in claim 16, wherein the planes are located such that posterior parts of the transparent material are not obscured by previously generated optical breakthroughs located in anterior parts of the transparent material parts.

18. A device for forming a cut which encloses a partial volume within a transparent material, said device comprising:
a source of laser radiation, which focuses laser radiation into the material at a focal point and causes optical breakthroughs substantially at the focal point along an optical axis,
a scanning unit, which three-dimensionally adjusts the focal point and
a control unit, which controls the scanning unit to form the cut by serial arrangement of the optical breakthroughs in the material, wherein the control unit adjusts the focal point along a spatial spiral, which is located in the desired location of the cut and extends along a main axis that is at substantially right angles to the optical axis.

19. The device as claimed in claim 18, wherein the scanning unit comprises adjustable optics for adjusting the focal point substantially along the optical axis and a deflecting unit for two-dimensional adjustment of the focal point substantially at right angles to the optical axis.

20. The device as claimed in claim 19, wherein the control unit controls the adjustable optics according to a substantially continuous, first substantially sinusoidal function.

21. The device as claimed in claim 20, wherein the control unit controls the deflecting unit in one of two spatial directions according to a second substantially sinusoidal function, and in the other of the two spatial directions according to a substantially linear function having an oscillation or stepped function superimposed thereon.

22. The device as claimed claim 18 wherein the control unit begins the spiral on a part of the transparent material which is posteriorly located substantially along the optical axis.

23. The device as claimed in claim 18, wherein the control unit arranges the main axis such that posterior parts of the transparent material are not obscured by previously created optical breakthroughs in anterior parts of the transparent material.

24. A device for forming a cut which encloses a partial volume within a transparent material, said device comprising:
a source of laser radiation, which focuses laser radiation into the material at a focal point and causes optical breakthroughs substantially at the focal point along an optical axis,
a scanning unit, which three-dimensionally adjusts the focal point and
a control unit, which controls the scanning unit to form the cut by serial arrangement of the optical breakthroughs in the material, wherein the control unit adjusts the focal point substantially along elevation lines, which are located in planes that are substantially parallel to the optical axis.

25. The device as claimed in claim 24, wherein the scanning unit comprises adjustable optics to adjust the focal point along the optical axis and a deflecting unit to effect two-dimensional adjustment of the focal point at substantially right angles to the optical axis.

26. The device as claimed in claim 25, wherein the control unit controls the adjustable optics according to a substantially continuous, first substantially sinusoidal function.

27. The device as claimed in claim 26, wherein the control unit controls the deflecting unit in one of two spatial directions according to a second substantially sinusoidal function, and in the other of the two spatial directions according to a substantially linear function having an oscillation or stepped function superimposed thereon.

28. The device as claimed claim 24, wherein the control unit begins the elevation line on a part of the transparent material which is posteriorly located substantially along the optical axis.

29. The device as claimed in claim 18, wherein the control unit arranges the planes such that posterior parts of the transparent material are not obscured by previously created optical breakthroughs located in anterior parts.
